(19) United States
(12) Patent Application Publication
(10) Pub. No.: US 2007/0298431 A1
(43) Pub. Date: Dec. 27, 2007

(54) Method of monitoring colorectal cancer

(76) Inventor: Volker Mai, Gainesville, FL (US)

Correspondence Address: Elizabeth Hart-Wells, Ph.D.;Room 021, 660 West Redwood Street, Baltimore, MD, 21201-1541, US

(21) Appl. No.: 11/820,429

(22) Filed: Jun. 19, 2007

Related U.S. Application Data
(60) Provisional application No. 60/815,126, filed on Jun. 20, 2006.

(51) Int. Cl. C12Q 1/68

(52) U.S. Cl. 435/6

(57) ABSTRACT

A method and kit is described for individualized stool surveillance for occurrence/recurrence of preneoplastic or neoplastic lesions based on the analysis of genetic mutations and methylation pattern detected in biopsy tissue removed during polypectomie as compared to normal colon mucosa.

This application claims the benefit of priority from U.S. Provisional Application Ser. No. 60/815,126 filed on Jun. 20, 2007

### INTRODUCTION

Epigenetic changes are now known to contribute to early steps in carcinogenesis and especially changes in methylation pattern of CpG islands in promoter regions of relevant genes have been well studied in colorectal carcinogenesis (Das and Singal, 2004, J. clin. Oncol. 22, 4632-42). Methylation pattern was found to significantly correlate in DNA extracted from colorectal mucosa and from fecal samples in the same individual (Belshaw et al., 2004, Cancer Epidemiol. Biomarkers Prev. 13, 1495-501). Methylation frequency of MGMT and CDKN4 and MLH1 differed in individuals with adenomas as compared to normal controls (Petko et al., 2005, Clin. Cancer Res. 11, 1203-9), characteristic methylation pattern were also detected in other cancers including esophagal adenocarcinoma (Eads et al., 2001, Cancer Res. 61, 3410-8).

It is assumed that aberrant methylation patterns detected in stool are due to shedding of cells from a preneoplastic lesion into the lumen. However, it is not known if methylation patterns in the promoter regions of these genes return to normal after polypectomie, which should result in the removal of the source for the aberrantly methylated DNA. If the patterns do disappear then a positive screening test for reappearance of the aberrant pattern over time might indicate the formation of new preneoplastic lesions.

Screening for colorectal cancer identifies preneoplastic lesions (polyps) in up to 25% of patients. Although such lesions are removed by polypectomie, patients are at an increased risk for developing additional polyps as well as colorectal cancer in the future. Surveillance is usually limited to repeat colonoscopies 5-10 years later, which results in some cancers that are not detected at an early curable stage.

Stool based detection of aberrant methylation might offer a superior opportunity to detect preneoplastic changes in relevant tissue (exfoliated colonocytes) even before mutations occur. Such screening tests might be especially useful for future active surveillance of polyp recurrence in subjects that have undergone polypectomie, as data on target regions for methylation analysis could be derived from the analysis of aberrant methylation patterns in polyp tissue.

I would like to implement efficient protocols for studying aberrant methylation patterns, as a marker of colorectal cancer (CRC) risk, in human DNA extracted from stool samples. Due to the presence of large amounts of bacterial DNA and inhibitors in stool, extraction of human DNA sufficiently clean for downstream applications is difficult. Capture probe based approaches have been used successfully, but their use is limited as only a few target DNA fragments can be isolated at a time (Petko et al., 2005, supra). Although successful use of a commercial kit has been described in one report (Belshaw et al., 2004, supra), we and others have not been able to repeat this consistently.

The proposed surveillance/screening test would detect early lesions that could be confirmed and removed by timely colonoscopy/polypectomie. This test could be administered yearly at moderate cost for detection of preneoplastic lesions. This test could also be utilized to monitor recurrence in subjects that underwent surgical removal of colorectal cancers.

A stool based genetic cancer screening test is currently commercially available for a set of preselected genes (EXACT SCIENCES, Boston, Mass.). Although this test has some utility as a generic screening test for an at average risk population it is not an efficient means for detecting recurrence of preneoplastic/neoplastic lesions. Our approach differs in that it is tailored towards an at-above risk population and based on genetic mutations and aberrant methylation pattern that will be detected in biopsy tissue from preneoplastic or neoplastic lesions of an individual. Identifying genetic/epigenetic lesions in the biopsy tissue will allow for the targeting of specific regions for the surveillance test, thus limiting costs while testing for highly specific changes that are likely to correlate with recurrence of lesions in an individual.

### SUMMARY OF THE INVENTION

We propose an individualized surveillance test for occurrence and/or recurrence of colorectal preneoplastic lesions based on identification of genetic mutations and aberrant methylation pattern detected in biopsy tissue removed by polypectomie during colorectal screening. DNA is extracted from polyp tissue to identify genes that are mutated and regions that are aberrantly methylated. Surveillance can then be individualized by screening stool samples for recurrence of the specific genetic/epigenetic signature detected in previously examined biopsy tissue from a preneoplastic lesion.

Therefore, it is an object of the present invention to provide a method and kit for monitoring colorectal cancer in a patient by identifying aberrant methylation pattern in nucleic acid of an adenomatomatous polyp from said patient and monitoring exfoliated colonocytes, or nucleic acid from colonocytes extracted from the stool, for recurrence of said aberrant pattern.

Methods of the invention are useful for detecting early-stage lesions in heterogeneous samples such as stool. Methods of the invention result in a high degree of sensitivity and specificity for the detection of early-stage disease. Methods of the invention are especially useful in detecting, for example, adenomas in the colon. Adenomas are non-metastatic lesions that frequently have the potential for metastasis. If all adenomas in a patient are detected and removed, the probability of complete cure is virtually certain.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1. COBRA analysis of MGMT in biopsy tissue Lanes 1-5: TaqI digestion, Lanes 6-10: undigested, M=marker, N=negative control. Partial digestion of the in vitro methylated is detected in lane 5.

FIG. 2. MSP analysis of MGMT methylation on native PAGE gel (M—marker (100 bp), Lanes 1 and 2 MGMT specific PCR (180 bp product), Lanes 3 and 4 Unmethylated MGMT specific PCR (93 bp product), N—negative control). PCR with primers against methylated MGMT did not yield any product (not shown).

### DETAILED DESCRIPTION

In a first aspect, the present invention relates to identifying an aberrant methylation pattern in a specific set of genes by analyzing nucleic acid from neoplastic polyps or lesions removed from a subject. The aberrant methylation can be in any part of the gene or genes, for example, the promoter, the transcribed sequence, the translated sequence. By aberrant methylation is meant reduction in methylation, increase in methylation, or change in methylation location as compared to normal tissue, e.g. normal colon mucosa.

In a second aspect of the invention, the present invention relates to a method for screening high-risk subjects for occurrence and/or recurrence of preneoplastic/neoplastic lesions. By high-risk subjects is meant a subject who has already had lesions or polyps removed by polypectomie, or a subject at an increased risk for developing additional polyps as well as colorectal cancer or a subject that has an increased risk for colorectal cancer due to preexisting conditions that include genetic predisposition and Inflammatory Bowel Disease.

Exfoliated colonocytes are isolated from stool and nucleic acid is isolated from these cells for analysis. According to the invention, nucleic acid can be a double-stranded DNA, single stranded DNA, RNA, or a nucleic acid analog.

The isolated nucleic acid can be analyzed for methylation pattern by methods known in the art. Methylation specific polymerase chain reaction (PCR) (MSP) of bisulfite DNA can be used for the detection of methylated CpG islands in the target nucleic acid. Optionally, methylation specific PCR can be combined with bisulfite restriction analysis. The nucleic acid sample may be treated with an agent such as sodium bisulfate, which modifies unmethylated cytosine to uracil without modifying methylated cytosine.

Primers for methylated and unmethylated DNA have been published (Petko et al., 2005, supra; Eads et al., 2001, supra). The primers can be targeted for five promoter regions that include CDKN2A, MGMT, MLH1, CALCA (calcitonin) and CDH1 (E-cadherin) for aberrant methylation analysis. Methylation pattern in the promoters of these genes were found to significantly correlate in DNA extracted from colorectal mucosa and from fecal samples in the same individual (Belshaw et al., 2004, supra). Aberrant methylation in CALCA and CDH1 has been shown in CRC. Methylation frequency of MGMT, CDKN4 and MLH1 differed in individuals with adenomas as compared to normal controls. Any one or more of the target genes can be analyzed for the presence of aberrant methylation. After identifying the aberrant methylation pattern, those genes containing the aberrant pattern, whether one or more, can be assayed individually or in combination, in order to determine the presence of aberrant methylation in stool samples.

Using the method of the present invention, other biological samples including sputum, blood, and other bodily fluids or tissues, that may or may not be mingled with other biological materials can be used for identifying aberrant methylation patterns leading to disease. These samples may contain nucleic acids indicative of a variey to diseases includig cancers, e.g. prostate, breast, lung, thymus, ovarian and so on.

Administration of the screening can be annually, or more or less frequently as the situation dictates.

The methods of the invention are useful for detecting diseases or disorders related to the colon including, but not limited to, cancer, pre-cancer and other diseases or disorders such as adenoma, polyp, inflammatory bowel disorder, inflammatory bowel syndrome, regional enteritis, granulomatous ileitis granulomatous ileocolitis, Crohn's Disease, ileitis, ileocolitis, jejunoileitis, granulomatous colitis, Yersinia enterocolitica enteritis, ulcerative colitis, psuedo-membraneous colitis, irritable bowel syndrome, diverticulosis, diverticulitis, intestinal parasites, infectious gastroenteritis, toxic gastroenteritis, and bacterial gastroenteritis.

The invention further relates to a diagnostic kit comprising one or more containers filled with one or more of the ingredient needed for sample collection and transport, one or more ingredient needed for extraction of nucleic acids from the biological sample, one or more ingredient needed for MSP and combined bisulfite restriction analysis, one or more primers targeted against genes of interest, and optionally one or more ingredient for quantifying methylation, which may include computer software. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

All publications, including, but not limited to patents and patent applications, cited in this specification are herein incorporated by reference as if each individual publication were specifically and individually indicated to be incorporated by reference herein as though fully set forth.

The invention is further described in detail to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided therein.

### EXAMPLE 1

We are currently performing a study in patients undergoing a screening colonoscopy at the UMMS GI clinic in which we enroll subjects after they have been referred for a colonoscopy, collect demographic and dietary data as well as a stool sample BEFORE the colonoscopy, obtain biopsy samples of normal colon tissue during the colonoscopy (as well as polyp tissue after pathology has been performed) and in a subset collect additional stool samples AFTER the colonoscopy/polypectomie. A total of 500 subjects are planned, so far we have completed data and sample collection in appr. 50 subjects.

Extraction of Human DNA from Feces

The largest proportion of DNA extracted from feces is of bacterial origin. In order to obtain sufficient material for methylation analysis of bisulfite converted or unconverted DNA by PCR we have optimized our extraction protocol. Instead of using bead beating for efficient cell lysis we are using a more gentle procedure that omits extensive enzymatic lysis steps. The Qiagen stool extraction kit contains an inhibitor matrix that binds and removes common PCR inhibitors. In order to concentrate the amount of human DNA we find it helpful to use Microcon YMC-100 spin columns. Although we have been able to extract human DNA from a subset of subjects, the success rate was below 50% and thus we clearly need a better method. We have recently been successful in obtaining PCR quality human DNA from 2/2 subjects with the use of the Somatic Cell Sampling and Recovery (SCSR™) kit (NonInvasive Technologies, Columbia Md.). SCSR™ is a proprietary cell isolation technology for obtaining somatic cells from fecal samples. We are currently working with the developer of this kit, located in the Baltimore area, to optimize the extraction protocol for our purpose. Even without optimization we were able to obtain up to >10⁶ exfoliated colonocytes and extracted sufficient amounts of PCR quality human DNA. In contrast to DNA extracted with the Qiagen kit we did not detect ANY inhibition when DNA was extracted with the SCSR™ kit. A further advantage of this kit is that it contains a transport medium that maintains the integrity of the colonocytes for more than seven days at room temperature, facilitating appropriate sample collection and transport.

Aberrant Methylation Analysis:

Methylation specific PCR (MSP) of bisulfite modified DNA is the method of choice for the detection of methylated CpG islands. Primers for methylated and unmethylated target DNA have been published (Petko et al., 2005, supra; Eads et al., 2001, supra), these primer set include Taqman based real time PCR assays.

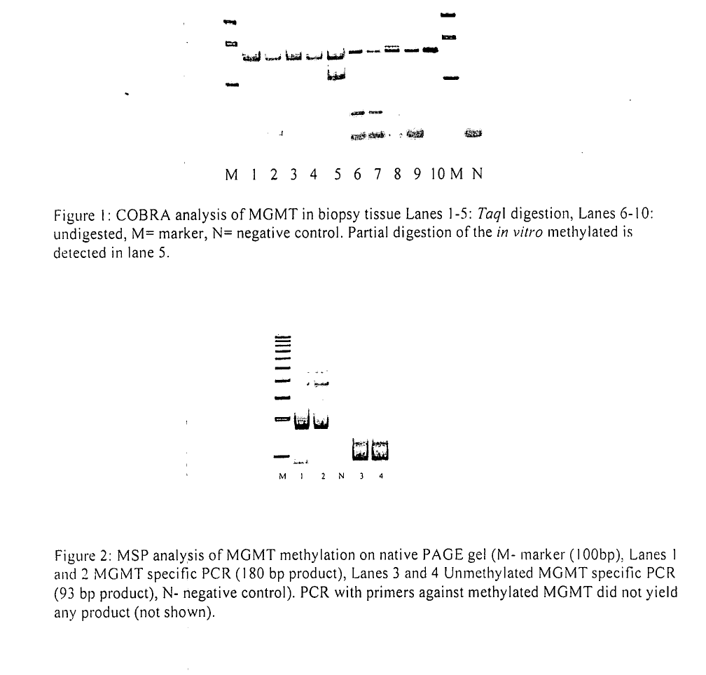

We have focused our initial experiments on using methylation specific PCR (MSP) and combined bisulfite restriction analysis (COBRA) with primers targeted against MGMT and DNA extracted from colon biopsies. We chose this initial target because differential methylation in MGMT has been shown in normal tissue from subjects without colonic lesions as compared to normal tissue adjacent to cancerous lesions (Shen et al., 2005, J. Natl. Cancer Inst. 97, 1330-8). This proposed field effect would increase the likelihood of detecting aberrant methylation in stool DNA in subjects with colonic lesions. We have optimized bisulfite conversion in the lab and now use the EZ DNA Methylation Gold-kit from Zymo Research (Orange, Calif.), which allows for an efficient conversion and yields sufficient amounts of clean converted DNA for MSP and COBRA analysis. Using biopsy DNA from four subjects and an in vitro methylated control DNA we performed COBRA analysis for the MGMT promoter (FIG. 1). Using DNA extracted from colon biopsies in two subjects without colonic disease we detected, as expected, unmethylated CpG islands in the MGMT gene (FIG. 2). We have also started to approach quantitation of methylation status using native PAGE gels to separate and quantify SYBR Gold stained restriction fragments.

Due to the co-purification of PCR inhibitors from stools we find it necessary to use Hotstart Taq (Qiagen) and add Q-mix (Qiagen) to our reactions in order to increase robustness of the PCR. MSP products are separated on a 6% PAGE gels and stained with SYBR Gold for improved sensitivity. Images are captured and analyzed with Quantity One Image software (Biorad), which can be used to quantify methylation by calculating the proportion of cut to uncut bands.

### EXAMPLE 2

To establish methods to reliably detect aberrant methylation pattern in stool samples Analysis of aberrant methylation pattern in stool DNA requires a robust protocol for DNA extraction followed by bisulfite conversion, (quantitative) amplification and restriction analysis.

DNA Extraction, Bisulfite Conversion and Methylation-Specific PCR Analysis from Paraffin-Embedded Proximal Colon Tissue

We have established a protocol to extract DNA from paraffin-embedded normal as well as polyp tissue. After removal of excessive paraffin by xylene and release of DNA from the blocks we follow the DNeasy extraction protocol for animal tissues (Qiagen). The extraction yields >0.5 μg of DNA >600 bp in length. This amount is sufficient for 4-8 bisulfite conversions with the EZ DNA kit (ZYMO Research).

Each bisulfite conversion yields sufficient template DNA for up to 10 methylation specific PCR reactions, allowing us to test a large panel of prospective markers without the need to multiplex the PCR reactions. We have been successful with this method in analyzing targets up to a length of 450 bp. We have had a 100% success rate with DNA extracted from paraffin tissue stored at room temperature for up to five years.

Our results indicate that we are able to use paraffin embedded tissues stored at room temperature for methylation specific PCR analysis to determine subject specific targets for polyp recurrence screening using normal as well as polyp tissue.

Successful extraction of DNA can be evaluated by PCR using primer sets directed against MyoD (550 bp product) and CoxII (95 bp product). Purified human DNA (Invitrogen) can serve as a positive control.

Extraction of DNA from Fecal Samples:

Although we already had some success with enrichment for exfoliated colonocytes, extraction of human DNA and subsequent methylation analysis using the SCSR™ kit we will evaluate another approach of enriching for exfoliated colonocytes that is based on the use of magnetic separation with Epithelial Enrich Dynabeads (Invitrogen, Calif.) that specifically bind epithelial cells. Important for our studies is the observation that the storage solution supplied with the NonInvasive kit maintains colonocyte integrity for at least seven days. Adding a magnetic separation step to the SCSR™ protocol is likely to improve purity of the DNA extracted from the isolated colonocytes.

Initial Target Regions for Methylation Analysis:

To optimize our protocols of methylation analysis we will initially use a set of five promoter regions that includes CDKN2A, MGMT, MLH1, CALCA and CDH1 for aberrant methylation analysis with DNA extracted from both biopsy and stool samples. Primers for methylated and unmethylated target DNA have been published. Methylation pattern in the promoters of these genes were found to significantly correlate in DNA extracted from colorectal mucosa and from fecal samples in the same individual (Belshaw et al., 2004, supra). Aberrant methylation in CALCA (calcitonin) and CDH1 (E-cadherin) has been shown in CRC. We are particularly interested in these two genes because calcitonin participates in calcium metabolism, which is associated with CRC risk and according to our own results from a recently completed pilot study high calcium intake correlates with higher numbers of beneficial lactic acid bacteria. E-cadherin is the target of the EBFT toxin, which is produced by a strain of the intestinal bacterium Bacteroides fragilis. Methylation frequency of MGMT, CDKN4 and MLH1 differed in individuals with adenomas as compared to normal controls, but no data is available on the frequency in African Americans. We will also evaluate the feasibility of using the TaqMan based assays that are published for our five target genes to better quantify methylation status.

We will use the Biocentre (CA) PCR optimization kit to determine the buffer system that works best with fecal human DNA and we will also evaluate enzymes other than Taq such as Tfl and Pfu as these enzymes might be more robust to stool contaminants.

### EXAMPLE 3

To Determine if Aberrant Methylation Pattern Detected in Polyp Tissue can be Detected in Pre- but not Post-Colonoscopy Stool Samples Collected from the Same Subject

It is assumed that the aberrant methylation patterns detected in stool are due to shedding of cells from a preneoplastic lesion into the lumen. It is not known if methylation pattern in the promoter regions of these genes return to normal after polypectomie, which should remove the source for the aberrantly methylated DNA. If the patterns do disappear than a positive screening test for reappearance of the aberrant pattern over time might indicate the formation of new preneoplastic lesions.

We will collect a stool sample BEFORE the colonoscopy, then obtain biopsy samples of normal colon tissue during the colonoscopy (as well as polyp tissue after pathology has been performed) and in addition collect stool sample AFTER the colonoscopy/polypectomie. For the studies proposed here we will choose subjects for which a polyp is removed during colonoscopy. As we don't know the outcome of the colonoscopy beforehand we will isolate colonocytes from stool samples of all subjects and freeze them until we have a diagnosis and access to the polyp tissue.

We are planning to initially identify ten subjects for which we can identify aberrant methylation pattern in any of the five target regions in polyp tissue. We will then proceed with analyzing methylation pattern in human DNA extracted from a normal biopsy sample and from stools collected before and after the polypectomie. Our analysis of methylation pattern in the five specific promoter regions will determine if polypectomie changes aberrant methylation pattern in stools of subjects for which such pattern can be detected in pre-colonoscopy stools but not in normal biopsies.

As a control we will also analyze methylation status in stool samples collected before and after colonoscopy and in normal colon biopsies in ten subjects for which no lesion is detected.

Our proposed study design is based on the assumption that we can identify ten subjects in which at least one of the five target genes is aberrantly methylated in polyp tissue. Although prior studies by others indicate that methylation in these regions is frequently observed in adenomatous polyps, we might have to expand our target set if we are not successful. A strength of our design that increases efficiency is that we will only analyze methylation in stool DNA AFTER we have already detected aberrant methylation in the same target region in polyp tissue from the same subject.

What is claimed is:

1. A method for monitoring recurrence of neoplastic polyps in a subject comprising,
(i) identifying aberrant methylation pattern in nucleic acid from adenomatous polyps removed from said subject,
(ii) testing nucleic acid from exfoliated colonocytes for said aberrant methylation pattern
wherein the presence of the aberrant methylation pattern in the exfoliated colonocytes predicts the recurrence of neoplastic polyps.

2. A method for identifying preneoplastic lesions in the colon of a subject comprising,
(i) testing nucleic acid from exfoliated colonocytes of said subject for presence of a methylation pattern different than a methylation pattern in normal colon mucosa from said subject,
wherein the presence of a different methylation pattern in the exfoliated colonocytes predicts the presence of preneoplastic polyps.

3. The method of claim 1 wherein the exfoliated colonocytes are found in a stool sample from said subject after polypectomie.

4. The method of claim 2 wherein the exfoliated colonocytes are found in a stool sample from said subject.

5. The method of claim 1 wherein said subject is a high risk subject.

6. The method of claim 5 wherein said high risk subject has a preexisting condition chosen from the group consisting of a subject that has undergone polypectomie, a subject with inflammatory bowel disease and a subject with genetic predisposition for colorectal cancer.

7. The method of claim 2 wherein said subject is a high risk subject.

8. The method of claim 7 wherein said high risk subject has a preexisting condition chosen from the group consisting of a subject that has undergone polypectomie, a subject with inflammatory bowel disease and a subject with genetic predisposition for colorectal cancer.
